SEQUENCE LISTING (2 sequences)

SEQ ID NO: 1 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 166 aa, Homo sapiens
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

(19) United States
(12) Patent Application Publication
(10) Pub. No.: US 2005/0220764 A1
(43) Pub. Date: Oct. 6, 2005

(54) Higher-doses of interferon-beta for treatment of multiple sclerosis

(75) Inventors: Ayad Abdul-Ahad, Basking Ridge, NJ (US); Dietmar Gross, White Plains, NY (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/815,342

(22) Filed: Apr. 1, 2004

(51) Int. Cl. A61K 38/21

(52) U.S. Cl. 424/85.6

(57) ABSTRACT

The present invention relates to pharmaceutical compositions comprising a new, therapeutically effective dose of an isolated interferon-beta (IFN-β) mutein for treatment of multiple sclerosis (MS) and methods of treating MS using such pharmaceutical compositions. More particularly, the pharmaceutical compositions of the present invention comprise a new, therapeutically effective dose of an isolated IFN-β mutein.

### FIELD OF THE INVENTION

The present invention is directed to pharmaceutical compositions comprising a new, therapeutically effective dose of an isolated interferon-beta (IFN-β) mutein for treatment of multiple sclerosis (MS) and methods of treating MS using such pharmaceutical compositions. More particularly, the pharmaceutical compositions of the present invention comprise a new, therapeutically effective dose of an isolated IFN-β mutein that is a variant of a native human IFN-β.

### BACKGROUND OF THE INVENTION

Multiple sclerosis (MS) is a chronic and severe disease characterized by focal inflammation in the central nervous system (CNS) (see e.g., Hemmer et al. (2002) Neuroscience 3: 291-301; Keegan at al. (2002) Ann. Rev. Med. 53: 285-302; Young, V. Wee (2002) Neurology 59: 802-808; Goodin at al. (2001) Am. Academy of Neurology 58: 169-178). An associated loss of the insulating myelin sheath from around the axons of the nerve cells (demyelination) and a degeneration of the axons are also prominent features of the disease. Resulting from the focal inflammation, an astrocytotic gliosis leads to the formation of sclerotic lesions in the white matter (see e.g., Prineas (1985) Demyelinating Diseases, Elsvevier: Amsterdam; Raine (1983) Multiple Sclerosis, Williams and Wilkins: Baltimore; Raine et al. (1988) J. Neuroimmunol. 20: 189-201; and Martin (1997) J. Neural Transmission (Suppl) 49: 53-67).

There are two major types of MS patient populations at the onset of the disease: those patients with relapsing-remitting MS and those patients with primary progressive MS. Relapsing-remitting MS is characterized by episodes (the so called relapses or exacerbation) where new neurologic deficits emerge or preexisting neurologic deficits worsen and periods of remission where the clinical symptoms are stabilized or diminished, whereas, primary progressive MS patients suffer from progressive neurological deterioration without exacerbations. A large proportion of patients with relapsing-remitting MS also experience during the course of their disease a worsening of neurologic symptoms independent of relapses, with or without superimposed relapses. Once this stage of the disease is reached, it is called secondary progressive MS.

The clinical symptoms of MS are thought to result from a focal breakdown in the blood-brain barrier (BBB) which permits the entry of inflammatory infiltrates into the brain and spinal cord. Further, these infiltrates are thought to consist of various lymphocytes and macrophages that lead to demyelination, axonal degeneration and scar tissue formation, and the degeneration of oligodendrocytes imperative to CNS myelin production (see e.g., Martin (1997) J. Neural Transmission (Suppl) 49:53-67). Consequently, the nerve-insulating myelin and the ability of oligodendroglial cells to repair damaged myelin are seriously compromised (see e.g., Scientific American 269(1993):106-114). These symptoms of MS include pain and tingling in the arms and legs, localized and generalized numbness, muscle spasm and weakness, difficulty with balance when standing or walking, difficulty with speech and swallowing, cognitive deficits, fatigue, and bowel and bladder dysfunction.

Although there is no known cure for MS, immunomodulatory therapy with interferons has proven to be successful in reducing the severity of the underlying disease in patients with MS. Interferons are important cytokines characterized by antiviral, antiproliferative, and immunomodulatory activities. These activities form a basis for the clinical benefits that have been observed in the treatment of patients with multiple sclerosis. The interferons are divided into the type I and type II classes. IFN-β belongs to the class of type I interferons, which also includes interferons alpha, tau and omega, whereas interferon gamma is the only known member of the distinct type II class.

Human IFN-β is a regulatory polypeptide with a molecular weight of 22 kDa consisting of 166 amino acid residues. The polypeptide can be produced by most cells in the body, in particular fibroblasts, in response to viral infection or exposure to other biologics. Further, IFN-β binds to a multimeric cell surface receptor, and productive receptor binding results in a cascade of intracellular events leading to the expression of IFNB inducible genes which in turn produces effects which can be classified as antiviral, antiproliferative and immunomodulatory.

Human IFN-β is a well-characterized polypeptide. The amino acid sequence of human IFN-β is known (see e.g., Gene 10:11-15,1980, and in EP 83069, EP 41313 and U.S. Pat. No. 4,686,191). Also, crystal structures have been reported for human and murine IFN-β, respectively (see e.g., Proc. Natl. Acad. Sci. USA 94:11813-11818, 1997. J. Mol. Biol. 253:187-207, 1995; reviewed in Cell Mol. Life Sci. 54:1203-1206, 1998). In addition, protein-engineered variants of IFN-β have been reported (see e.g., WO 9525170, WO 9848018, U.S. Pat. No. 5,545,723, U.S. Pat. No. 4,914,033, EP 260350, U.S. Pat. No. 4,588,585, U.S. Pat. No. 4,769,233, Stewart et al, DNA Vol. 6 No. 2 1987 pp. 119-128, Runkel et al, 1998, Jour. Biol. Chem. 273, No. 14, pp. 8003-8008). Also, the expression of IFN-β in CHO cells has been reported (see e.g., U.S. Pat. No. 4,966,843, U.S. Pat. No. 5,376,567 and U.S. Pat. No. 5,795,779).

Further, IFN-β molecules with a particular glycosylation pattern and methods for their preparation have been reported (see e.g., EP 287075 and EP 529300). Also reported is the modification of polypeptides by polymer conjugation or glycosylation. For example, polymer modification of native IFN-β or a C17S variant thereof has been reported (see e.g., EP 229108, U.S. Pat. No. 5,382,657, EP 593868, U.S. Pat. No. 4,917,888 and WO 99/55377). Pegylated lysine depleted polypeptides have also been reported, wherein at least one lysine residue has been deleted or replaced with any other amino acid residue (see e.g., U.S. Pat. No. 4,904,584). Further processes for conjugating a protein with PEG have been reported (see e.g., WO 99/67291), wherein at least one amino acid residue on the protein is deleted and the protein is contacted with PEG under conditions sufficient to achieve conjugation to the protein.

Pegylated variants of polypeptides belonging to the growth hormone superfamily have also been reported, wherein a cysteine residue has been substituted with a non-essential amino acid residue located in a specified region of the polypeptide and IFN-β has been reported as one example of a polypeptide belonging to the growth hormone superfamily (see e.g., WO 99/03887). Glycosylated and pegylated IFN-β are reported e.g., in WO 00/23114. WO 00/26354 reports a method of producing a glycosylated polypeptide variant with reduced allergenicity, which as compared to a corresponding parent polypeptide comprises at least one additional glycosylation site.

Also reported is the modification of granulocyte colony stimulating factor (G-CSF) and other polypeptides so as to introduce at least one additional carbohydrate chain as compared to the native polypeptide (see e.g., U.S. Pat. No. 5,218,092). IFN-β is mentioned as one example among many polypeptides that allegedly can be modified according to the technology described in U.S. Pat. No. 5,218,092.

Further, IFN-β fusion proteins are reported, e.g., in WO 00/23472.

Commercial preparations of IFN-β are approved for the treatment of patients with MS and are sold under the names Betaseron® (also termed Betaferon® or IFN-β 1bₛₑᵣ₁₇, which is non-glycosylated, produced using recombinant bacterial cells, has a deletion of the N-terminal methionine residue and the C17S mutation), Avonex® and Rebif® (also termed IFN-β1a, which is glycosylated, produced using recombinant mammalian cells. Further, a comparison of IFN-β 1a and IFN-β 1b with respect to structure and function has been presented in Pharm. Res. 15:641-649, 1998.

IFN-β is the first therapeutic intervention shown to delay the progression of MS. In addition, the approved dose of IFN-β has been shown to be effective in reducing the exacerbation rate of MS, and more patients remain exacerbation-free for prolonged periods of time as compared with placebo-treated patients. Furthermore, the accumulation rate of disability is reduced (see e.g., Neurol. 51:682-689,1998).

IFN-β has inhibitory effects on the proliferation of leukocytes and antigen presentation. Furthermore, IFN-β may modulate the profile of cytokine production towards an anti-inflammatory phenotype. Finally, IFN-β can reduce T-cell migration by inhibiting the activity of T-cell matrix metalloproteases. Such IFN-β activities are likely to act in concert to account for the beneficial effect of IFN-β in the treatment of patients with MS (see e.g., Neurol. 51:682-689, 1998).

The currently approved interferons are regarded by the United States Food and Drug Administration (US FDA) as effective and well-tolerated in the treatment of patients with MS. However, these therapeutic agents are only partially effective because they are able to slow the rate of disease progression of MS and but not arrest progression or cure the disease. Thus, there is a well-recognized need for an MS drug treatment with higher efficacy.

### SUMMARY OF THE INVENTION

The present invention provides pharmaceutical compositions comprising a new, therapeutically effective dose of an isolated interferon-beta (IFN-β) mutein for treatment of MS and methods of treating MS using such pharmaceutical compositions. Preferably, the new, therapeutically effective amount of IFN-β mutein is greater than 250 mcg, and more preferably, greater than 375 mcg. In one aspect, the new, therapeutically effective amount of IFN-β is at least about 375 mcg to at least about 500 mcg, or at least about 500 mcg to at least about 625 mcg. In another aspect, the new, therapeutically effective amount of IFN-β is at least about 450 mcg to at least about 550 mcg, or at least about 475 mcg to at least about 525 mcg. In another aspect, the new, therapeutically effective amount is about 500 mcg.

The isolated IFN-β mutein of the present invention is preferably a synthetic or recombinant IFN-β polypeptide, and can be a variant of a biologically active, native IFN-β, e.g., human IFN-β and, more preferably, human IFNβ-1b. In particular, the isolated IFN-β mutein of the present invention can be a human IFN-β mutein and, more preferably, Betaseron® (also termed Betaferon® or IFN-β 1bₛₑᵣ₁₇). The pharmaceutical compositions of the present invention can be stabilized, human serum albumin-free (HSA-free) pharmaceutical compositions. More particularly, the stabilized, HSA-free pharmaceutical compositions of the present invention can comprise an IFN-β mutein that is substantially monomeric and solubilized in a low-ionic-strength formulation.

In particular, the isolated IFN-β mutein of the present invention can be a variant of a biologically active, native IFN-β where: 1) the native IFN-β has at least one cysteine residue that is free to form a disulfide link and is nonessential to the biological activity of the native IFN-β;2) the amino acid positions of the IFN-β mutein are numbered in accordance with the native IFN-β; and 3) the IFN-β mutein has at least one cysteine residue deleted or replaced by another amino acid residue, and exhibits the biological activity of native IFN-β. Further, the isolated IFN-β mutein of the present invention can have other modifications. For example the IFN-β mutein of the present invention can lack an N-terminal methionine, may or may not be glycosylated, and/or may have a secretion signal sequence or other additional sequences e.g., fused thereto.

In one aspect, the invention provides a pharmaceutical composition having IFN-β activity and comprising a new, therapeutically effective amount of an isolated IFN-β mutein, wherein the therapeutically effective amount is in a range that is greater than 375 mcg to at least about 500 mcg, and wherein the IFN-β mutein has a cysteine at position 17 deleted or replaced by a neutral amino acid. In another aspect, the new, therapeutically effective amount is at least about 500 to about at least about 625 mcg. In another aspect, the new, therapeutically effective amount is at least about 450 mcg to at least about 550 mcg. In another aspect, the new, therapeutically effective amount is at least about 475 to at least about 525 mcg. In one aspect, the new, therapeutically effective amount is about 500 mcg.

In another aspect, the isolated IFN-β mutein of the present invention has a neutral amino acid that is selected from a group consisting of serine, threonine, glycine, alanine, valine, leucine, isoleucine, histidine, tyrosine, phenylalanine, tryptophan, and methionine. In one aspect, the neutral amino acid is serine.

In another aspect, the isolated IFN-β mutein of the present invention lacks an N-terminal methionine.

In one aspect, the invention provides a pharmaceutical composition having IFN-β activity and comprising a new, therapeutically effective amount of an isolated IFN-β mutein, wherein the new, therapeutically effective amount is about 500 mcg, and wherein the isolated IFN-β mutein is a variant of a human IFN-β where a cysteine at position 17 is replaced by a serine.

In another aspect, the isolated IFN-β mutein of the present invention is Betaseron®).

In one aspect, the present invention provides a stabilized, HSA-free pharmaceutical composition comprising an IFN-β mutein.

In another aspect, the stabilized, HSA-free pharmaceutical composition of the present invention comprises an IFN-β mutein that is substantially monomeric and solubilized in a low-ionic-strength formulation. In a related aspect, the low-ionic-strength formulation is a solution having a pH from about 2 to about 5, and an ionic strength from about 1 mM to about 100 mM.

In one aspect, the present invention provides a method of treating a patient for multiple sclerosis comprising administering to the patient a pharmaceutical composition of the present invention (as described herein).

### BRIEF DESCRIPTION OF THE DRAWINGS

The foregoing and other objects of the present invention, the various features thereof, as well as the invention itself may be more fully understood from the following description, when read together with the accompanying drawings in which:

FIG. 1 is a schematic illustrating the Betaseron dose escalation scheme using 250 mcg or higher-dose 500 mcg Betaseron in MS patients over a period of 12 weeks (see Example 1).

FIG. 2 is a schematic illustrating the baseline demographics of the MS patients randomized to either a 250 mcg or higher-dose 500 mcg Betaseron dosing regimen (see Example 1).

FIG. 3 is a schematic illustrating that, with respect to primary outcome adverse events (AEs), higher-dose 500 mcg Betaseron is safe and well-tolerated as compared to the 250 mcg dose of Betaseron (see Example 1).

FIG. 4 is a schematic illustrating that the dose escalation scheme for higher-dose 500 mcg Betaseron is at least as successful as the dose escalation scheme for 250 mcg Betaseron in MS patients, where over 90% of the patients attained the full, higher-dose 500 mcg Betaseron during the course of the study (see Example 1).

FIG. 5 is a schematic illustrating the median percent change from baseline (BL) of the T2 lesion number in MS patients receiving 250 mcg dose and higher-dose 500 mcg Betaseron (see Example 1).

FIG. 6 is a schematic illustrating the median percent change from baseline (BL) of the T2 lesion volume in MS patients receiving 250 mcg dose and higher-dose 500 mcg Betaseron (see Example 1).

### DETAILED DESCRIPTION OF THE INVENTION

The present invention provides pharmaceutical compositions comprising a new, therapeutically effective amount of an isolated interferon-beta (IFN-β) mutein for use in the treatment of multiple sclerosis (MS). The present invention further provides methods of treating MS using such a pharmaceutical composition comprising a new, therapeutically effective amount of an IFN-β mutein. The new, therapeutically effective amount is greater than the standard dose of interferons currently approved for use in the treatment of MS. Preferably, a new, therapeutically effective amount of IFN-β mutein is greater than 250 mcg, and more preferably, greater than 375 mcg. In one embodiment, the new, therapeutically effective amount of IFN-β mutein is at least about 375 mcg to at least about 625 mcg. In another embodiment, the new, therapeutically effective amount of the IFN-β mutein is at least about 625 mcg to at least about 1000 mcg. In other embodiments, the new, therapeutically effective amount of IFN-β is at least about 375 mcg to at least about 500 mcg, or at least about 500 mcg to at least about 625 mcg. In some embodiments, the new, therapeutically effective amount of IFN-β is at least about 450 mcg to at least about 550 mcg, or at least about 475 mcg to at least about 525 mcg. In another embodiment, the new, therapeutically effective amount is about 500 mcg.

The standard dose of IFN-β (e.g., Betaseron®) approved for use in the treatment of MS is 250 mcg. However, the maximum therapeutically effective dose of IFN-β has not previously been known. Also, it has not previously been known whether higher doses of IFN-β lead to improved efficacy for treatment of a patient with MS. Further, previous studies by others teach that doses higher than the approved amount are not well-tolerated in MS patients (see e.g., Knobler et al., J. Interferon Res. (1993) 13: 333-340). However, contrary to the teachings of others, the present invention provides a new, higher therapeutically effective amount of an IFN-β mutein that is safe, well-tolerated and shows a positive trend towards beneficial effects for use in the treatment of patients with MS, and this dose is higher than the approved, standard dose of IFN-β. Thus, the pharmaceutical compositions and methods of the present invention can increase the possibility of benefits from treatment of MS using IFN-β and, also, the number of patients that benefit from treatment.

The references cited herein, including patents and patent applications, are incorporated by reference, in their entirety.

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Standard reference works setting forth the general principles of recombinant DNA technology include Sambrook, J., et al. (1989) Molecular Cloning: A Laboratory Manual, 2d Ed., Cold Spring Harbor Laboratory Press, Planview, N.Y.; McPherson, M. J. Ed. (1991) Directed Mutagenesis: A Practical Approach, IRL Press, Oxford; Jones, J. (1992) Amino Acid and Peptide Synthesis, Oxford Science Publications, Oxford; Austen, B. M. and Westwood, O. M. R. (1991) Protein Targeting and Secretion, IRL Press, Oxford. Any suitable materials and/or methods known to those of ordinary skill in the art can be utilized in carrying out the present invention. However, preferred materials and methods are described. Materials, reagents and the like to which reference is made in the following description and examples are obtainable from commercial sources, unless otherwise noted.

Treatment of Multiple Sclerosis

The pharmaceutical compositions and methods of the present invention are for use in the treatment of patients suffering from various clinically recognized forms of MS, including but not limited to, relapsing-remitting MS, different types of progressive MS (including, but not limited to, e.g., primary and secondary progressive MS, progressive-relapsing MS) and, also, clinically isolated syndromes suggestive of MS.

As used herein, “relapsing-remitting” MS is a clinical course of MS that is characterizedve MS, progressive-relapsing MS) and, also, clinically isolated syndromes suggestive of MS.

As used herein, “relapsing-remitting” MS is a clinical course of MS that is characterized by clearly defined, sporadic exacerbations or relapses, during which existing symptoms become more severe and/or new symptoms appear. Such exacerbations or relapses, may be followed by partial recovery, or full recovery and remission. The length of time between these sporadic exacerbations or relapses may be months or years, during which time inflammatory lesions, demyelination, axonal loss, and scar formation may still proceed. Relapsing-remitting MS is the most common beginning phase of MS, and it has been reported that about 50% of the cases having progression within 10 to 15 years, and another 40% within 25 years of onset.

As used herein, “primary-progressive” MS is a clinical course of MS that is characterized from the beginning by progressive disease, with no plateaus or remissions, or an occasional plateau and very short-lived, minor improvements. As the disease progresses, the patient may experience difficulty in walking, the steadily decline in motor skills, and an increase in disabilities over many months and years, generally, in the absence of those distinct inflammatory attacks characteristic of relapsing-remitting MS.

As used herein, “secondary-progressive” MS is a clinical course of MS that initially is relapsing-remitting and then becomes progressive at a variable rate independent of relapses. Although patients experiencing this type of MS may continue to experience inflammatory attacks or exacerbations, eventually the exacerbations and periods of remission may diminish, with the disease taking on the characteristic decline observed with primary-progressive MS.

As used herein “progressive-relapsing” MS is a clinical course of MS that may show permanent neurological deterioration from the onset of the disease, but with clear, acute exacerbations or relapses that look like relapsing-remitting MS. For these patients, lost functions may never return. It has been reported that this type of MS has a high mortality rate if untreated.

Clinically isolated syndromes suggestive of MS include, but are not limited to, early onset multiple sclerosis and monosymptomatic MS. For purposes of the present invention, the term “multiple sclerosis” is intended to encompass each of these clinical manifestations of the disease and clinically isolated syndromes suggestive of MS unless otherwise specified.

As used herein, a “therapeutically effective dose” or “therapeutically effective amount” of an IFN-β mutein of the present invention is a dose or amount, when administered to a patient with MS as described herein, provides for treatment of MS.

As used herein, “treating” or “treatment” of MS using the pharmaceutical compositions and methods of the present invention result in an improvement in the disease or associated symptoms in a patient with MS. Thus, when a patient suffering from MS undergoes treatment in accordance with the pharmaceutical compositions and methods of the present invention, treatment can result in the prevention and/or amelioration of MS disease symptoms, disease severity, and/or periodicity of recurrence of the disease, i.e., treatment of MS using the compositions and methods of the present invention can result in lengthening the time period between episodes in which symptoms flare, and/or can suppress the ongoing immune or autoimmune response associated with the disease, which, left untreated, can enhance disease progression and disability.

As used herein “patient” refers to a subject, preferably a human, who is in need of treatment. For example, a subject having MS or symptoms associated with MS is a patient in need of treatment of MS or associated symptoms of MS. A patient can be pre-treated for MS with a pharmaceutical composition or can be a naive patient who has not been pre-treated for MS with a pharmaceutical composition, prior to treatment with the higher-dose pharmaceutical composition or methods of the present invention. For example, a pre-treated patient can be one who has been pretreated with a different amount of an interferon or IFN-β mutein, e.g., a standard approved dose (e.g., 250 mcg Betaseron) prior to treatment with the higher-dose pharmaceutical compositions or methods of the present invention. For example, an approved dose of Betaseron®, Avonex®, or Rebif®) can be used to pre-treat patients. The pharmaceutical compositions and methods of the present invention are suitable for use in the treatment of pre-treated and naive patients.

Factors influencing the amount of IFN-β mutein that constitutes a therapeutically effective dose include, but are not limited to, the severity of the disease, the history of the disease, and the age, health, and physical condition of the individual undergoing therapy. Generally, a higher-dose of this therapeutic agent (i.e., IFN-β mutein of the present invention) is preferred as tolerated and safe. As used herein, a “new” or “higher” therapeutically effective amount of an IFN-β mutein refers to a therapeutically effective amount of an IFN-β mutein of the present invention that is greater than the standard approved dose for treatment of MS (i.e., 250 mcg) and preferably, greater than 375 mcg to about at least 500 mcg. Preferably, a higher, therapeutically effective amount of IFN-β mutein is greater than 250 mcg, and more preferably, greater than 375 mcg. In one embodiment, the higher, therapeutically effective amount of IFN-β mutein is at least about 375 mcg to at least about 625 mcg. In another embodiment, the higher, therapeutically effective amount of the IFN-β mutein is at least about 625 mcg to at least about 1000 mcg. In other embodiments, the higher, therapeutically effective amount of IFN-β is at least about 375 mcg to at least about 500 mcg, or at least about 500 mcg to at least about 625 mcg. In some embodiments, the higher, therapeutically effective amount of IFN-β is at least about 450 mcg to at least about 550 mcg, or at least about 475 mcg to at least about 525 mcg. In another embodiment, the higher, therapeutically effective amount is about 500 mcg. Further, a higher, therapeutically effective dose of an IFN-β mutein of the present invention can also depend upon the dosing frequency and severity of the disease in the MS patient undergoing treatment.

In a preferred embodiment, the higher, therapeutically effective dose of an IFN-β mutein of the present invention can be administered subcutaneously with a dosing frequency of every other day. In another embodiment, the dosing frequency can be once to twice a week, three to four times a week, or five to six times a week, or daily.

The dosing regimen can be continued for as long as is required to achieve the desired effect, i.e., for example, prevention and/or amelioration of the disease, symptoms associated with the disease, disease severity, and/or periodicity of the recurrence of the disease, as described herein. In one embodiment, the dosing regimen is continued for a period of up to one year to indefinitely, such as for one month to 30 years, about three months to about 20 years, about 6 months to about 10 years.

Symptoms of MS that are prevented, ameliorated, or treated, when a patient undergoes therapy in accordance with the methods of the present invention, include, e.g.,: weakness and/or numbness in one or more extremities; tingling of the extremities and tight band-like sensations around the trunk or limbs; tremor of one or more extremities; dragging or poor control of one or both legs to spastic or ataxic paraparesis; paralysis of one or more extremities; hyperactive tendon reflexes; disappearance of abdominal reflexes; Lhermitte's sign; retrobulbar or optic neuritis; unsteadiness in walking; increased muscle fatigue; brain stem symptoms (diplopia, vertigo, vomiting); disorders of micturition; hemiplegia; trigeminal neuralgia; other pain syndromes; nystagmus and ataxia; cerebellar-type ataxia; Charcot's triad; diplopia; bilateral internuclear ophthalmoplegia; myokymia or paralysis of facial muscles; deafness; tinnitus; unformed auditory hallucinations (because of involvement of cochlear connections); transient facial anesthesia or of trigeminal neuralgia; bladder dysfunction euphoria; depression; fatigue; dementia, dull, aching pain in the low back; sharp, burning, poorly localized pains in a limb or both legs and girdle pains; abrupt attacks of neurologic deficit; dysarthria and ataxia; paroxysmal pain and dysesthesia in a limb; flashing lights; paroxysmal itching; and/or tonic seizures, taking the form of flexion (dystonic) spasm of the hand, wrist, and elbow with extension of the lower limb. A patient having MS may have one or more of the symptoms associated with MS and one or more can be ameliorated by the pharmaceutical compositions and methods of the present invention.

The pharmaceutical compositions disclosed herein can also block or reduce the physiological and pathogenic deterioration associated with MS, e.g., inflammatory response in the brain and other regions of the nervous system, breakdown or disruption of the blood-brain barrier, appearance of lesions in the brain, tissue destruction, demyelination, autoimmune inflammatory response, acute or chronic inflammatory response, neuronal death, and/or neuroglial death. Beneficial effects of the pharmaceutical compositions and methods of the present invention include, e.g., preventing the disease, slowing the onset of established disease, ameliorating symptoms of the disease, reducing the annual exacerbation rate (i.e., reducing the number of episodes per year), slowing the progression of the disease, or reducing the appearance of brain lesions (e.g., as identified by MRI scan), and postponing or preventing disability including cognitive decline, loss of employment, hospitalization, and finally death. The episodic recurrence of a particular type of MS can be ameliorated, e.g., by decreasing the severity of the symptoms (such as the symptoms described above) associated with the, e.g., MS episode, or by lengthening the time period between the occurrence of episodes, e.g., by days, weeks, months, or years, where the episodes can be characterized by the flare-up and exacerbation of disease symptoms, or preventing or slowing the appearance of brain inflammatory lesions (see, e.g., Adams (1993) Principles of Neurology, page 777, for a description of a neurological inflammatory lesion).

Adverse effects due to some MS treatment regimens are known in the art (see, e.g., Munschauer et al. (1997) Clinical Therapeutics 19(5): 883-893; Walther et al. (1999) Neurology 53: 1622-1627; Lublin et al. (1996) 46: 12-18; Bayas et al. (2000) 2: 149-159; Ree et al. (2002) 8: 15-18; Walther et al. (1998) 5(2): 65-70). For example, some of the adverse effects due to treatment of MS include, but are not limited, e.g., flu-like symptoms; increased spasticity or deterioration of neurological symptoms; menstrual disorders; laboratory abnormalities (e.g., abnormal blood count/value for hemoglobin, leukocytes, granulocytes, lymphocytes, or thrombocytes); abnormal laboratory value for liver enzymes (e.g. bilirubin, transaminases, or alkaline phosphatases); injection site reactions, (e.g., inflammation, pain, or erythema); cutaneous or subcutaneous necroses; and depression. Suitable co-medications and the use of these co-medications for treating adverse effects due to treatment of MS can be determined according to co-medications generally known in the art for treatment of such effects (see, e.g., Munschauer et al. (1997) Clinical Therapeutics 19(5): 883-893; Walther et al. (1999) Neurology 53: 1622-1627; Lublin et al. (1996) 46: 12-18; Bayas et al. (2000) 2: 149-159; Ree et al. (8: 15-18; Walther et al. (1998) 5(2): 65-70). Doses and dosing regimens for such co-medications are also generally known. Examples of such co-medications include, but are not limited to, analgesics, steroids, and non-steroidal anti-inflammatory drugs (NSAIDs).

Suitable examples of co-medications also include, but are not limited to, e.g., ibuprofen, acetaminophen, acetylsalicyclic acid, prednisone, pentoxifylline, baclofen, steroids, antibacterial agents, and antidepressants (see e.g., Walther et al. (1999) Neurology 52: 1622-1627). For example, flu-like symptoms can be treated with NSAIDs (e.g., ibuprofen or acetylsalicylic acid) or with paracetamol or with pentoxifylline; increased spasticity or deterioration of neurological symptoms can also be treated with NSAIDs and/or muscle relaxants (e.g., baclofen); menstrual disorders can be treated with oral contraceptives; injection site reactions can be treated with systemic NSAIDs and/or steroids (e.g., hydrocotisone); cutaneous or subcutaneous necrosis can be treated with antibacterial agents and depression can be treated with antidepressants (see e.g., Walther et al. (1999) Neurology 53: 1622-1627).

Combination therapies with other drugs, which are effective in the treatment of MS and have a different adverse event profile may increase the treatment effect and level out the adverse event profile. Suitable examples of combination therapies include, but are not limited to, e.g., glatiramer acetate (Copaxone), mitoxantrone, cyclophosphamide, cyclosporine A, cladribine, monoclonal antibodies (e.g., Campath-H1® or Antegren G/Natazulimab®), and statins.

Effective treatment of MS in a patient using the methods of the invention can be examined in several alternative ways including, for example, EDSS (extended disability status scale) score, Functional Composite Score, cognitive testing, appearance of exacerbations, or MRI.

The EDSS is a means to grade clinical impairment due to MS (see e.g., Kurtzke (1983) Neurology 33:1444). Eight functional systems, the walking range, the ability to walk, and the ability to maintain self-care functions are evaluated for the type and severity of neurologic impairment. For example, prior to treatment, impairment in the following systems is evaluated: pyramidal, cerebellar, brainstem, sensory, bowel and bladder, visual, cerebral, and other. Together with the assessment of the walking range, of the ability to walk with or without assistive devices, and of the ability to maintain self-care functions the final EDSS score is calculated. Follow-up scores are then obtained at defined intervals of treatment. The grade scale may range, e.g., from 0 (normal) to 10 (death due to MS). An increase of one full step (or a one-half step at the higher baseline EDSS scores) may define disease progression (see e.g., Kurtzke (1994) Ann. Neurol. 36:573-79, Goodkin (1991) Neurology. 41:332.).

Exacerbations can be defined as the appearance of a new symptom that is attributable to MS and accompanied by an appropriate new neurologic abnormality (see e.g., IFN-β MS Study Group). Exacerbations typically last at least 24 hours, and are preceded by stability or improvement for at least 30 days or a separation of at least 30 days from onset of the last event. Standard neurological examinations may result in the exacerbations being classified as either mild, moderate, or severe according to changes in a Neurological Rating Scale (see e.g., Sipe et al. (1984) Neurology 34:1368), and/or changes in EDSS score or evaluating physician opinion. An annual exacerbation rate (or other measures for the frequency of relapses, like e.g., a hazard ratio for recurrent relapses), the proportion of exacerbation-free patients, and other relapse-based measures for disease activity are then determined, and the effectiveness of therapy is assessed between the treated group and the placebo group, for any of these measurements. A number of new technologies can also be used to diagnose and manage MS. For example, magnetic resonance imaging (MRI) scanning can be used as a concomitant indicator of disease and disease activity, and can also be used as a diagnostic tool (see e.g., Paty et al (1993) Neurology 43: 662-667; Frank et al. (1994) Ann. Neurology 36(suppl.): S86-S90; The IFN-β Multiple Sclerosis Study Group (1995) Neurology 45: 1277-1285; Filippi et al. (1994) Neurology 44: 635-641). For example, MRI can be used to measure active lesions using, e.g., gadolinium-DTPA-enhanced T1-weighted imaging (see e.g., McDonald et al. (2001) Ann. Neurol. 50: 121-127) or the location and extent of lesions using T2-weighted and T1-weighted techniques. For example, baseline MRIs are obtained and thereafter, the same imaging plane and patient position are used for each subsequent study. Areas of lesions can be outlined and summed slice by slice for total lesion area. Various criteria may be examined, e.g.,: 1) evidence of new lesions; 2) rate of appearance of active or new lesions; and 3) change in lesion area or lesion volume (see e.g., Paty et al. (1993) Neurology 43:665). Improvement due to therapy may then be established, e.g., when there is a statistically significant improvement in an individual patient compared to baseline or in a treated group versus a placebo group.

Interferon-Beta Muteins

As used herein, “IFN-β mutein” or “interferon-beta mutein” refers to variants of a native IFN-β and can also be referred to as IFN-βb-like polypeptides. Preferably, the IFN-β mutein is a human IFN-β mutein. Variants of native human IFN-β which may be naturally occurring (e.g., allelic variants that occur at the IFN-β locus) or recombinantly or synthetically produced, have amino acid sequences that are similar to, or substantially similar to a mature native IFN-β sequence. An example of an amino acid sequence of a mature native human IFN-β is SEQ ID NO: 1. IFN-β muteins also encompass fragments of IFN-β or truncated forms of IFN-β that retain IFN-β activity. These biologically active fragments or truncated forms of IFN-β can be generated by removing amino acid residues from the full-length IFN-β amino acid sequence using recombinant DNA techniques well known in the art. IFN-β muteins of the present invention may be glycosylated or not glycosylated.

The IFN-β muteins of the present invention also muteins of a mature human, native IFN-β sequence (e.g., IFN-β 1b), wherein one or more cysteine residues that are not essential to IFN-β biological activity have been deliberately deleted or replaced with other amino acids to eliminate sites for either intermolecular crosslinking or incorrect intramolecular disulfide bond formation. IFN-β muteins of this type include those containing a glycine, valine, alanine, leucine, isoleucine, tyrosine, phenylalanine, histidine, tryptophan, serine, threonine, or methionine substituted for the cysteine found at amino acid 17 of the mature native IFN-β amino acid sequence. Serine and threonine are the more preferred replacements because of their chemical analogy to cysteine. Serine substitutions are most preferred. An example of an amino acid sequence of an IFN-β mutein of the present invention is SEQ ID NO: 2. In a preferred embodiment, the IFN-β mutein is Betaseron® (see e.g., U.S. Pat. Nos. 4,588,585; 4,959,314; 4,737,462; L. Lin (1998) Dev. Biol. Stand. 96: 97-104).

In one embodiment, the cysteine found at amino acid 17 of the mature native sequence is replaced with serine. Cysteine 17 may also be deleted using methods known in the art (see, for e.g., U.S. Pat. No. 4,588,585), resulting in an IFN-β mutein that is one amino acid shorter than the mature native IFN-β (see also, e.g., U.S. Pat. Nos. 4,530,787; 4,572,798; and 4,588,585). Thus, IFN-β muteins with one or more mutations that improve the therapeutic utility of an IFN-β are encompassed by the present invention.

Additional changes can be introduced by mutation into the nucleotide sequences encoding IFN-β thereby leading to changes in the IFN-β amino acid sequence, without altering the biological activity of the interferon. Thus, an isolated nucleic acid molecule encoding an IFN-β mutein having a sequence that differs from the amino acid sequence for the mature native IFN-β can be created by introducing one or more nucleotide substitutions, additions, or deletions into the corresponding nucleotide sequence (see, e.g., U.S. Pat. No. 5,588,585), such that one or more amino acid substitutions, additions or deletions are introduced into the encoded IFN-β. Mutations can be introduced by standard techniques, such as site-directed mutagenesis and PCR-mediated mutagenesis. Such IFN-β muteins are also encompassed by the present invention.

As an example, conservative amino acid substitutions may be made at one or more predicted, preferably nonessential amino acid residues. As used herein, a “nonessential” amino acid residue is a residue that can be altered from the wild-type sequence of IFN-β without altering its biological activity, whereas an “essential” amino acid residue is required for biological activity. As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In preferred embodiments, such substitutions are not made for conserved amino acid residues, or for amino acid residues residing within a conserved motif.

Alternatively, IFN-β mutein nucleotide sequences can be made by introducing mutations randomly along all or part of an IFN-β coding sequence, such as by saturation mutagenesis, and the resultant mutants can be screened for IFN-β biological activity to identify mutants that retain activity. Following mutagenesis, the encoded protein can be expressed recombinantly, and the activity of the protein can be determined using standard assay techniques described herein.

In preferred embodiments, biologically active IFN-β muteins have at least 80%, more preferably about 90% to about 95% or more, and most preferably about 96% to about 99% or more amino acid sequence identity to the amino acid sequence of a mature, native IFN-β which serves as the basis for comparison or reference. As used herein “sequence identity” is the same amino acid residues that are found within the variant polypeptide and the polypeptide molecule that serves as a reference when a specified, contiguous segment of the amino acid sequence of the variant is aligned and compared to the amino acid sequence of the reference molecule.

For the optimal alignment of two sequences for the purposes of sequence identity determination, the contiguous segment of the amino acid sequence of the mutein may have additional amino acid residues or deleted amino acid residues with respect to the amino acid sequence of the reference molecule. The contiguous segment used for comparison to the reference amino acid sequence will comprise at least 20 contiguous amino acid residues. Corrections for increased sequence identity associated with inclusion of gaps in the amino acid sequence of the mutein can be made by assigning gap penalties. Methods of sequence alignment are well known in the art.

For example, the determination of percent identity between any two sequences can be accomplished using a mathematical algorithm. One preferred, non-limiting example of a mathematical algorithm utilized for the comparison of sequences is e.g., the algorithm of Myers and Miller (1988) Comput. AppI. Biosci. 4:11-7. Such an algorithm is utilized in the ALIGN program (version 2.0), which is part of the GCG alignment software package. A PAM120 weight residue table, a gap length penalty of 12, and a gap penalty of 4 can be used with the ALIGN program when comparing amino acid sequences. Another preferred, non-limiting example of a mathematical algorithm for use in comparing two sequences is the algorithm of Karlin and Altschul (1990) Proc. Natl. Acad. Sci. USA 90:5873-5877, modified as in Karlin and Altschul (1993) Proc. Natl. Acad. Sci USA 90:5873-5877. Such an algorithm is incorporated into the NBLAST and XBLAST programs of Altschul et al. (1990) J. Mol. Biol. 215:403-410. BLAST amino acid sequence searches can be performed with the XBLAST program, score=50, wordlength=3, to obtain amino acid sequence similar to the polypeptide of interest.

To obtain gapped alignments for comparison purposes, gapped BLAST can be utilized as described in Altschul et al. (1997) Nucleic Acids Res. 25:3389-3402. Alternatively, PSI-BLAST can be used to perform an integrated search that detects distant relationships between molecules (see e.g., Altschul et al. (1997) supra.). When utilizing BLAST, gapped BLAST, or PSI-BLAST programs, the default parameters can be used (see e.g., www.ncbi.nim.nih.gov). Also see the ALIGN program (Dayhoff (1978) in Atlas of Protein Sequence and Structure 5:Suppl. 3, National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art (see, e.g., Myers and Miller (1988) Comput. Appl. Biosci. 4:11-17).

Biologically active IFN-β muteins encompassed by the present invention also include IFN-β muteins that are covalently linked with, e.g., polyethylene glycol (PEG) or albumin. These covalent hybrid IFN-β molecules can have certain desirable pharmaceutical properties such as an extended serum half-life after administration to a patient. Methods for creating PEG-IFN adducts involve chemical modification of monomethoxypolethylene glycol to create an activated compound that will react with IFN-β. Methods for making and using PEG-linked polypeptides are reported, e.g., in Delgado et al. (1992) Crit. Rev. Ther. Drug. Carrier Syst. 9:249-304 (and as described herein in the Background). Methods for creating albumin fusion polypeptides involve fusion of the coding sequences for the polypeptide of interest (e.g., IFN-β) and albumin and are reported, e.g., in U.S. Pat. No. 5,876,969.

Biologically active IFN-β muteins encompassed by the invention preferably retain IFN-β activities, particularly the ability to bind to IFN-β receptors. In some embodiments, the IFN-β mutein retains at least about 25%, about 50%, about 75%, about 85%, about 90%, about 95%, about 98%, about 99% or more of the biologically activity of the reference IFN-β polypeptides. IFN-β muteins whose activity is increased in comparison with the activity of the reference polypeptides are also encompassed. The biological activity of IFN-β variants can be measured by any method known in the art (see e.g., assays described in Fellous et al. (1982) Proc. Natl. Acad. Sci USA 79:3082-3086; Czerniecki et al. (1984) J. Virol. 49(2):490-496; Mark et al. (1984) Proc. Natl Acad. Sci. USA 81:5662-5666; Branca et al. (1981) Nature 277:221-223; Williams et al. (1979) Nature 282:582-586; Herberman et al. (1979) Nature 277:221-223; Anderson et al. (1982) J. Biol. Chem. 257(19):11301-11304).

Suitable IFN-β muteins for use in the pharmaceutical compositions and methods of the present invention can be variants of a native IFN-β of any mammalian species including, but not limited to, avian, canine, bovine, porcine, equine, and human. Preferably, the IFN-β mutein of the present invention is a variant of a native human IFN-,8, in either its glycosylated or unglycosylated form. Most preferably, the IFN-β mutein of the present invention is a variant of human IFN-β 1b.

Non-limiting examples of IFN-β muteins encompassed by the invention are set forth in, e.g., Nagata et al. (1980) Nature 284:316-320; Goeddel et al. (1980) Nature 287:411-416; Yelverton et al. (1981) Nucleic Acids Res. 9:731-741; Streuli et al. (1981) Proc. Natl. Acad. Sci. U.S.A. 78:2848-2852; EP028033B1, and EP109748B1. See also, e.g., U.S. Pat. Nos. 4,518,584; 4,569,908; 4,588,585; 4,738,844; 4,753,795; 4,769,233; 4,793,995; 4,914,033; 4,959,314; 5,545,723; and 5,814,485. These citations also provide guidance regarding residues and regions of the IFN-β polypeptide that can be altered without the loss of biological activity.

In some embodiments of the present invention, the IFN-β is recombinantly produced. As used herein “recombinantly produced” IFN-β is IFN-β that has comparable biological activity to mature native IFN-β and that has been prepared by recombinant DNA techniques. IFN-β can be produced by culturing a host cell transformed with an expression vector comprising a nucleotide sequence that encodes an IFN-β polypeptide. The host cell is one that can transcribe the nucleotide sequence and produce the desired protein, and can be prokaryotic (see, e.g., E. coli) or eukaryotic (e.g., a yeast, insect, or mammalian cell). Examples of recombinant production of IFN-β, including suitable expression vectors, are provided in, e.g., Mantei et al. (1982) Nature 297:128; Ohno et al. (1982) Nucleic Acids Res. 10:967; Smith et al.. (1983) Mol. Cell. Biol. 3:2156, and U.S. Pat. No. 4,462,940, 5,702,699, and 5,814,485; herein incorporated by reference. Also, e.g., see U.S. Pat. No. 5,795,779, where IFN-β is recombinantly produced in Chinese hamster ovary (CHO) cells.

Human interferon genes have been cloned using recombinant DNA (“rDNA”) technology and have been expressed in E. coli (see e.g., Nagola et al. (1980) Nature 284:316; Goeddel et al. (1980) Nature 287:411; Yelverton et al. (1981) Nuc. Acid Res. 9:731; Streuli et al. (1981) Proc. NatI. Acad. Sci. U.S.A. 78:2848). Alternatively, IFN-β can be produced, e.g., by a transgenic animal or plant that has been genetically engineered to express the IFNβ protein of interest in accordance with methods known in the art.

Proteins or polypeptides that exhibit native IFN-β-like properties may also be produced with rDNA technology by extracting poly-A-rich 12S messenger RNA from virally induced human cells, synthesizing double-stranded cDNA using the mRNA as a template, introducing the CDNA into an appropriate cloning vector, transforming suitable microorganisms with the vector, harvesting the microorganisms, and extracting the interferon-beta therefrom (see, e.g., European Patent Application Nos. 28033 (published May 6, 1981); 32134 (published Jul. 15, 1981); and 34307 (published Aug. 26, 1981)), which describe various methods for the production of IFN-β employing rDNA techniques.

Alternatively, the IFN-β mutein of the present invention can be synthesized chemically, by any of several techniques that are known to those skilled in the peptide art (see e.g., Li et al. (1983) Proc. Natl. Acad. Sci. USA 80:2216-2220, Steward and Young (1984) Solid Phase Peptide Synthesis (Pierce Chemical Company, Rockford, Ill.), and Baraney and Merrifield (1980) The Peptides: Analysis, Synthesis, Biology, ed. Gross and Meinhofer, Vol. 2 (Academic Press, New York, 1980), pp. 3-254, discussing solid-phase peptide synthesis techniques; and Bodansky (1984) Principles of Peptide Synthesis (Springer-Verlag, Berlin) and Gross and Meinhofer, eds. (1980), The Peptides. Analysis, Synthesis, Biology, Vol. 1 (Academic Press, New York, discussing classical solution synthesis). The IFNβ mutein of the present invention can also be chemically prepared by the method of simultaneous multiple peptide synthesis. See, for example, Houghten (1984) Proc. Natl. Acad. Sci. USA 82:5131-5135; and U.S. Pat. No. 4,631,211.

Pharmaceutical Compositions

The pharmaceutical compositions of the present comprise a higher therapeutically effective amount of an IFN-β mutein, and are suitable for use in the methods of the present invention. Methods for formulating pharmaceutical compositions are generally known in the art. For example, see Remington's Pharmaceutical Sciences 18.sup.th ed.: Mack Pub. Co.: Eaton, Pa. 1990, for a thorough discussion on the formulation and selection of pharmaceutically acceptable carriers, stabilizers, and isomolytes. Also, for formulating pharmaceutical compositions comprising IFN-β or IFN-β muteins, see, e.g., U.S. Pat. Nos. 4,588,585, 5,183,746; 5,795,779; and 5,814,485; U.S. Application Nos. 10/190,838, 10/035,397; and PCT International Application Nos. PCT/US02/21464 and PCT/US01/51074.

A pharmaceutically acceptable carrier may be used in combination with the IFN-β mutein and other components (e.g., co-medications) in the pharmaceutical compositions of the present invention. As used herein, “pharmaceutically acceptable carrier” is a carrier or diluent that is conventionally used in the art to facilitate the storage, administration, and/or the desired effect of the therapeutic ingredients of the pharmaceutical composition. A carrier may also reduce any undesirable side effects of the therapeutic agent, e.g., the IFN-β mutein of the present invention. A suitable carrier is preferably stable, e.g., incapable of reacting with other ingredients in the formulation. Further, a suitable carrier preferably does not produce significant local or systemic adverse effect in recipients at the dosages and concentrations employed for therapy. Such carriers are generally known in the art.

Suitable pharmaceutically acceptable carriers are, e.g., solvents, dispersion media, antibacterial and antifungal agents, microcapsules, liposomes, cationic lipid carriers, isotonic and absorption delaying agents and the like which are not incompatible with the active or therapeutic ingredients (e.g., an IFN-β mutein of the present invention) of the pharmaceutical compositions of the present invention. The use of such media and agents for therapeutically effective or active substances is well known in the art. Supplementary active ingredients may also be incorporated into the pharmaceutical compositions of the present invention and used in the methods of the present invention.

Additional examples of pharmaceutically suitable carriers for use in the pharmaceutical compositions of the present invention are large stable macromolecules such as albumin, gelatin, collagen, polysaccharide, monosaccarides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), heparin alginate, and the like. Slow-release carriers, such as hyaluronic acid, may also be suitable.

Stabilizing agents such as human serum albumin (HSA), mannitol, dextrose, trehalose, thioglycerol, and dithiothreitol (DTT), may also be added to the pharmaceutical compositions of the present invention to enhance their stability. Suitable stabilizing agents include but are not limited to ethylenediaminetetracetic acid (EDTA) or one of its salts such as disodium EDTA; polyoxyethylene sorbitol esters e.g., polysorbate 80 (TWEEN 80), polysorbate 20 (TWEEN 20); polyoxypropylene-polyoxyethylene esters e.g., Puronic F68 and Pluronic F127; polyoxethylene alcohols e.g., Brij 35; semethicone; polyethylene glycol e.g., PEG400; lysophosphatidylcholine; and polyoxyethylene-p-t-octyphenol e.g., Triton X-100. Stabilization of pharmaceutical compositions by surfactants is generally known in the art (see e.g., Levine et al.. (1991) J. Parenteral Sci. Technol. 45(3):160-165).

Other acceptable components of the pharmaceutical compositions of the present invention may include, but are not limited to, buffers that enhance isotonicity such as water, saline, phosphate, citrate, succinate, acetic acid, aspartate, and other organic acids or their salts. Preferably, pharmaceutical compositions of the present invention comprise a non-ionic tonicifying agent in an amount sufficient to render the compositions isotonic with body fluids. The pharmaceutical compositions of the present invention can be made isotonic with a number of non-ionic tonicity modifying agents generally known to those in the art, e.g., carbohydrates of various classifications (see, e.g., Voet and Voet (1990) Biochemistry (John Wiley & Sons, New York); monosaccharides classified as aldoses (e.g., glucose, mannose, arabinose), and ribose, as well as those classified as ketoses (e.g., fructose, sorbose, and xylulose); disaccharides (e.g., sucrose, maltose, trehalose, and lactose); and alditols (acyclic polyhydroxy alcohols) e.g., glycerol, mannitol, xylitol, and sorbitol. In a preferred embodiment, non-ionic tonicifying agents are trehalose, sucrose, and mannitol, or a combination thereof.

Preferably, the non-ionic tonicifying agent is added in an amount sufficient to render the formulation isotonic with body fluids. In one embodiment, when incorporated into a pharmaceutical composition of the present invention (including, e.g., an HSA-free pharmaceutical composition), the non-ionic tonicifying agent is present at a concentration of about 1% to about 10%, depending upon the agent used (see e.g., U.S. application Ser. Nos. 10/190,838, 10/035,397; and PCT International Application Nos. PCT/US02/21464 and PCT/US01/51074).

Other acceptable components of the pharmaceutical compositions of the present invention may include, but are not limited to, e.g., co-medications. Such co-medications are well known in the art and may include, but are not limited to, e.g., those that help alleviate or mitigate adverse effects due to MS or due to treatment of the disease. Such co-medications include, but are not limited to, e.g., analgesics, non-steroidal anti-inflammatory drugs (NSAIDs), and steroids, as discussed herein and generally known in the art.

Further, preferred pharmaceutical compositions of the present invention may incorporate buffers having reduced local pain and irritation resulting from injection, or improve solubility or stability of a component (e.g., an IFN-β mutein) of the pharmaceutical compositions of the present invention. Such buffers include, but are not limited to, e.g., low-phosphate, aspartate, and succinate buffers.

In one embodiment, the pharmaceutical compositions of the present invention comprise a higher, therapeutically effective amount of a pharmaceutical composition that is stabilized and HSA-free. As used herein an “HSA-free” pharmaceutical composition refers to a pharmaceutical composition prepared in the absence of HSA and is thus free of this pharmaceutical excipient. Preferably, the stabilized, HSA-free pharmaceutical compositions of the present invention comprise a higher, therapeutically effective amount of an IFN-β mutein that is substantially monomeric and solubilized in a low-ionic-strength formulation. As used herein, “substantially monomeric” IFN-β mutein refers to where the majority of the IFN-β mutein (by weight) in a composition is a monomer and not aggregated e.g., as a dimer, trimer or other multimer. As used herein, “solubilized” IFN-β mutein refers to the IFN-β mutein that is soluble in solution and not precipitated out of solution. Preferably, the low-ionic-strength formulation has an advantage of stabilizing the IFN-β mutein and maintaining the IFN-β mutein in solution in substantially monomeric form. As used herein, a “stabilized” pharmaceutical composition of the present invention (e.g., a stabilized, HSA-free pharmaceutical composition), refers to a pharmaceutical composition of the present invention where the IFN-β mutein is substantially monomeric when in solution and is suitable for use in the compositions and methods of the present invention.

As used herein a “low-ionic-strength” formulation is a solution that comprises a concentration of the buffer sufficient to maintain the buffer at low ionic strength, preferably in a range from about 1 mM to about 100 mM. In a preferred embodiment, the low-ionic-strength formulation is a solution having a pH from about 2 to about 5, and an ionic strength from about 1 mM to about 100 mM. Suitable buffers for preparation of the low-ionic-strength formulation include, but are not limited to, e.g., glycine, aspartic acid glutamic acid, sodium succinate, formate, acetate, citrate, phosphate, histidine, and imidazole. The stabilized and HSA-free compositions and low-ionic-strength formulations of the present invention can be prepared according to methods known in the art (see, e.g., U.S. Pat. Nos. 4,588,585, 5,183,746; 5,795,779; and 5,814,485; U.S. application Ser. Nos. 10/190,838, 10/035,397; and PCT International Application Nos. PCT/US02/21464 and PCT/US01/51074).

The pharmaceutical composition may additionally comprise a solubilizing compound or formulation that is capable of enhancing the solubility of the IFN-β mutein of the present invention. Suitable solubilizing compounds include, e.g., compounds containing a guanidinium group, preferably arginine. Additional examples of suitable solubilizing compounds include, but are not limited to, e.g., the amino acid arginine, or amino acid analogues of arginine that retain the ability to enhance the solubility of an IFN-β mutein of the present invention. Examples of contain arginine. Further examples of suitable solubilizing compounds are discussed in, e.g., U.S. Pat. Nos. 4,816,440; 4,894,330; 5,005,605; 5,183,746; 5,643,566; and in Wang et al. (1980) J. Parenteral Drug Assoc. 34: 452-462)

In preferred embodiments, the pharmaceutical compositions of the present invention comprise IFN-β mutein formulated in a unit dosage and in an injectable form such as a solution, suspension, or emulsion, or in the form of lyophilized powder, which can be converted into solution, suspension, or emulsion prior to administration. The pharmaceutical compositions of the present invention may be sterilized by membrane filtration, which also removes aggregates, and stored in unit-dose or multi-dose containers such as sealed vials, ampules or syringes.

Liquid, lyophilized, or spray-dried pharmaceutical compositions comprising IFN-β or IFN-β mutein may be prepared as known in the art, e.g., as an aqueous or nonaqueous solution or suspension for subsequent administration to a patient in accordance with the methods of the present invention. Each of these pharmaceutical compositions may comprise an IFN-β mutein as a therapeutically or prophylactically effective or active component. As used herein, a therapeutically or prophylactically “effective” or “active” component is an IFN-β mutein that is included in the pharmaceutical composition of the present invention to bring about a desired therapeutic or prophylactic response with regard to treatment, prevention, or diagnosis of an MS disease or condition in a patient having MS, using the pharmaceutical compositions and methods of the present invention. Preferably the pharmaceutical compositions of the present invention comprise appropriate stabilizing agents, bulking agents, or both to minimize problems associated with loss of protein stability and biological activity during preparation and storage.

Formulation of the IFN-β mutein for use in the pharmaceutical compositions and methods of the present invention are preferably stable under the conditions of manufacture and storage and preserved against the contaminating action of microorganisms such as bacteria and fungi. Methods of preventing microorganism contamination are well known, and can be achieved e.g., through the addition of various antibacterial and antifungal agents.

Suitable forms of the pharmaceutical composition of the present invention may include sterile aqueous solutions or dispersions, and sterile powders for the extemporaneous preparation of sterile injectable solutions or dispersion. Suitable forms are preferably sterile and fluid to the extent that they can easily be taken up and injected via a syringe. Typical carriers may include a solvent or dispersion medium containing, for example, water buffered aqueous solutions (i.e., biocompatible buffers), ethanol, polyols such as glycerol, propylene glycol, polyethylene glycol, suitable mixtures thereof, surfactants, or vegetable oils. Sterilization can be accomplished by any art-recognized technique, including but not limited to filtration or addition of antibacterial or antifungal agents, for example, paraben, chlorobutanol, phenol, sorbic acid or thimerosal. Further, isotonic agents such as sugars or sodium chloride may be incorporated in the subject compositions.

Production of sterile injectable solutions containing an IFN-β mutein of the present invention may be accomplished by incorporating the polypeptide in the desired amount, in an appropriate solvent with various ingredients (e.g., those enumerated herein) as desired, and followed by sterilization. To obtain a sterile powder, the above solutions can be vacuum-dried or freeze-dried as necessary.

The IFN-β mutein of the present invention can thus be compounded for convenient and effective administration in pharmaceutically effective amounts with a suitable pharmaceutically acceptable carrier in a therapeutically effective dose.

The precise therapeutically effective amount of an IFN-β mutein to be used in the compostions and methods of the present invention for application to humans can be determined by the skilled artisan with consideration of individual differences in age, weight, extent of cellular infiltration by inflammatory cells and condition of the MS patient. Preferably, a higher, therapeutically effective amount of IFN-β mutein is greater than 250 mcg, and more preferably, greater than 375 mcg. In one embodiment, the higher, therapeutically effective amount of IFN-β mutein is at least about 375 mcg to at least about 625 mcg. In another embodiment, the higher, therapeutically effective amount of the IFN-β mutein is at least about 625 mcg to at least about 1000 mcg. In other embodiments, the higher, therapeutically effective amount of IFN-β is at least about 375 mcg to at least about 500 mcg, or at least about 500 mcg to at least about 625 mcg. In some embodiments, the higher, therapeutically effective amount of IFN-β is at least about 450 mcg to at least about 550 mcg, or at least about 475 mcg to at least about 525 mcg. In another embodiment, the higher, therapeutically effective amount is about 500 mcg.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier.

The principal active ingredients (e.g., an IFN-β mutein of the present invention and, optionally, a co-medication) may be compounded for convenient and effective administration in therapeutically effective amounts with a suitable pharmaceutically acceptable carrier in dosage unit form as described herein. A unit dosage form can, for example, contain the principal active compound (i.e., an IFN-β mutein of the present invention) in a higher, therapeutically effective amount that is preferably greater than 250 mcg, and more preferably, greater than 375 mcg. In one embodiment, the higher, therapeutically effective amount of IFN-β mutein is at least about 375 mcg to at least about 625 mcg. In another embodiment, the higher, therapeutically effective amount of the IFN-β mutein is at least about 625 mcg to at least about 1000 mcg. In other embodiments, the higher, therapeutically effective amount of IFN-β is at least about 375 mcg to at least about 500 mcg, or at least about 500 mcg to at least about 625 mcg. In some embodiments, the higher, therapeutically effective amount of IFN-β is at least about 450 mcg to at least about 500 mcg, or at least about 475 mcg to at least about 525 mcg. In another embodiment, the higher, therapeutically effective amount is about 500 mcg.

The co-medications are contained in a unit dosage form in amounts generally known in the art. In the case of compositions containing supplementary active ingredients, e.g. co-medications, the dosages may be determined, e.g., by reference to the known dose and manner of administration of the ingredients.

Packaging material used to contain the active ingredient (i.e., the IFN-β mutein) of the pharmaceutical composition of the present invention can comprise glass, plastic, metal or any other suitable inert material and, preferably, is packaging material that does not chemically react with any of the ingredients contained therein.

The pharmaceutical compositions of the present invention may be administered in a manner compatible with the dosage formulation and in such an amount as will be therapeutically effective. Further, the pharmaceutical compositions of the present invention may be administered in any way which is medically acceptable and which may depend on the specific MS type or associated symptoms being treated. Possible administration routes include injections, by parenteral routes such as intravascular, intravenous, intra-arterial, subcutaneous, intramuscular, intratumor, intraperitoneal, intraventricular, intraepidural or others, as well as oral, nasal, ophthalmic, rectal, topical, or by inhalation. In a preferred embodiment, the administration route is subcutaneous.

Sustained release administration is also contemplated, e.g., using erodible implants.

In a preferred embodiment, the pharmaceutical composition of the present invention comprises a therapeutically effective amount of IFN-β mutein that is greater than 250 mcg, more preferably, greater than 375 mcg, and most preferably greater than 375 mcg to at least about 500 mcg, at least about 500 mcg to at least about 625 mcg. In another embodiment, the therapeutically effective amount of IFN-β mutein is at least about 625 mcg to at least about 1000 mcg.

In another preferred embodiment, the IFN-β mutein of the present invention is a purified, sterile, lyophilized protein product produced by recombinant DNA techniques and formulated for use by subcutaneous injection. For example, the IFN-β mutein can be manufactured by bacterial fermentation of a strain of E. coli that carrys a plasmid encoding the mutein. In a preferred embodiment, the IFN-β mutein is human interferon beta-1bₛₑᵣ₁₇ (i.e., Betaseron®/Betaferon®).

In another preferred embodiment, IFN-β 1bₛₑᵣ₁₇ is 165 amino acids in length, has a molecular weight of approximately 18,500 daltons. In another preferred embodiment, the IFN-β 1bₛₑᵣ₁₇ polypeptide is made by isolating the native human IFN-β 1b gene from human fibroblasts and substituting the serine at position 17 with cysteine. In another preferred embodiment, the specific activity of IFN-β 1bₛₑᵣ₁₇ is approximately 32 million international units (IU)/mg. In a preferred embodiment, Betaseron®) (IFN-β 1bₛₑᵣ₁₇) is supplied as a lyophilized powder containing a higher therapeutically effective amount of IFN-β1bₛₑᵣ₁₇, and human albumin USP (United States Pharmacopoeia) and mannitol USP as stabilizers. In one embodiment, the stabilizers are human albumin USP and dextrose USP. In one preferred embodiment, the lyophilized protein product is a sterile, white to off-white powder that is intended for subcutaneous injection after reconstitution with a diluent supplied (e.g., the diluent can be a sodium chloride solution, preferably a 54% solution of sodium chloride).

In a preferred embodiment, the protein product is packaged in a clear glass, single-use vial; and a separate vial containing diluent (e.g., a 0.54% solution of sodium chloride) is included for each vial of drug. In another preferred embodiment, the diluent is provided in a syringe (i.e., the syringe is pre-filled with the diluent). In yet another preferred embodiment, the pharmaceutical composition of the present invention is provided in solution in a syringe (i.e., the syringe is pre-filled with the pharmaceutical composition in solution) and is ready for use.

In a preferred embodiment, the pharmaceutical composition of the present invention can be stored under refrigeration, between 2° to 8° C. (36° to 46° F.). In another embodiment, the pharmaceutical composition is stored at room temperature.

In a preferred embodiment, the pharmaceutical composition of the present invention is administered subcutaneously, every other day. In another preferred embodiment, the subcutaneous administration is via automated or manual injection (e.g., using a syringe) of the pharmaceutical composition.

The invention is further illustrated by the following examples that are not intended in any way to limit the scope of the invention.

### EXAMPLES

### Example 1

This Example illustrates the safety, tolerability, and positive trend towards beneficial effects of 500 mcg versus 250 mcg Betaseron (IFN-β 1bₛₑᵣ₁₇) administered subcutaneously every other day (eod) in naive MS patients. The effects of 500 mcg versus 250 mcg of Betaseron were measured by magnetic resonance imaging (MRI) criteria, including gadolinium enhancing lesion number and combined unique lesion activity, in patients with relapsing-remitting MS. Using MRI parameters to monitor the effects of higher-dose Betaseron in the treatment of patients with MS, the findings of this study indicate a positive trend towards the beneficial effects of 500 mcg Betaseron as compared to the currently approved 250 mcg dose of Betaseron. Thus, the results of this study demonstrate the safe, well-tolerated, and positive trend towards beneficial effects of administering 500 mcg subcutaneous dose IFN-β 1bₛₑᵣ₁₇ eod to patients with RRMS.

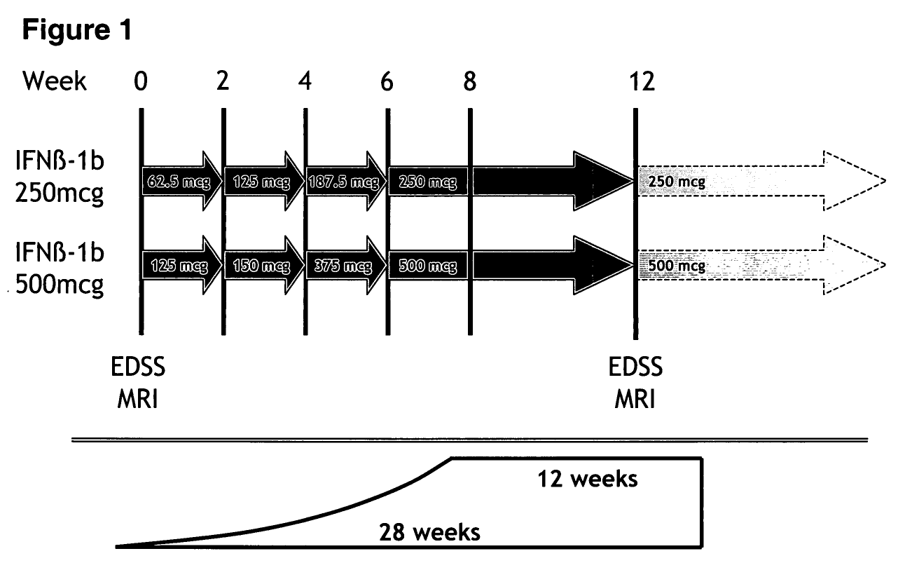

Design/Methods: A multicenter, randomized, double-blind, parallel group study comparing Betaseron (IFN-β 1bₛₑᵣ₁₇) 500 mcg with 250 mcg, self-administered by subcutaneous injection eod for at least 12 weeks. Patients were instructed to use auto-injectors to give consistency of injection technique. The Betaseron was escalated over the first 6 to 12 weeks, and then maintained at full-dose for the duration of the study until the last randomized patient finished 12 weeks of treatment (see FIG. 1). Non-steroidal anti-inflammatory drugs were administered concomitantly with Betaseron injections to minimize flu-like symptoms. The safety and tolerability of the drug at the 500 mcg and 250 mcg dose was defined by the proportion of patients in each treatment arm experiencing flu-like syndrome, fever, myalgia, injection site reactions, asthenia, headache, and liver and bone marrow function abnormalities.

The first phase of this study compared the effect of Betaseron at doses of 250 mcg and 500 mcg on various brain MRI measures including the frequency of enhancing lesions and combined unique lesion activity. All patients also underwent gadolinium enhanced (0.1 mmol/kg) MRI scanning at baseline and week 12 according to a standardized protocol, and the MRI scans were analyzed in a blinded fashion.

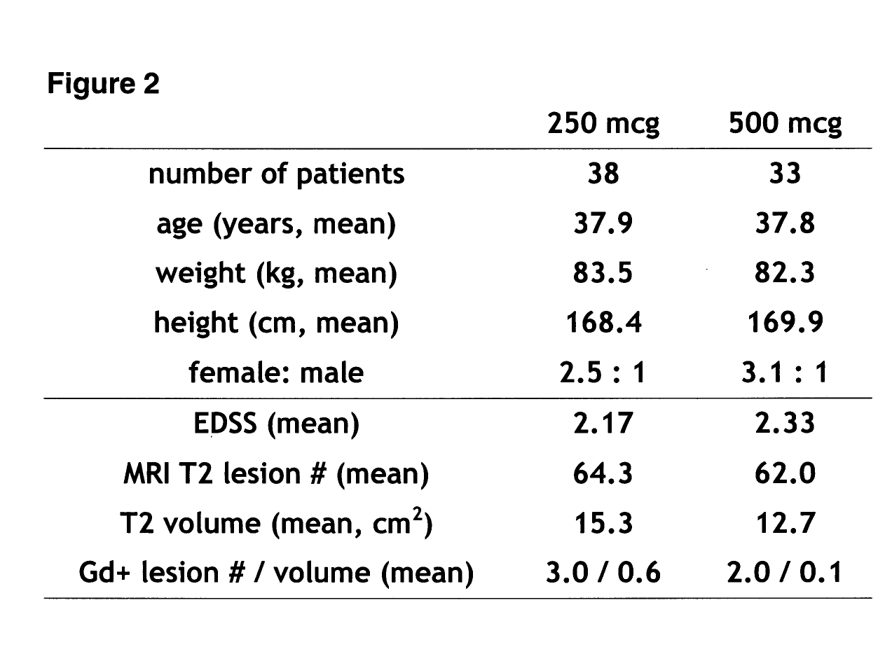

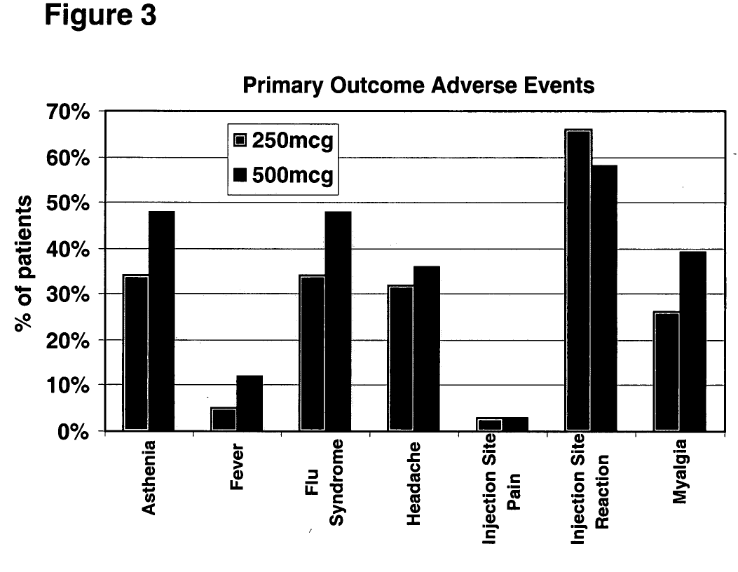

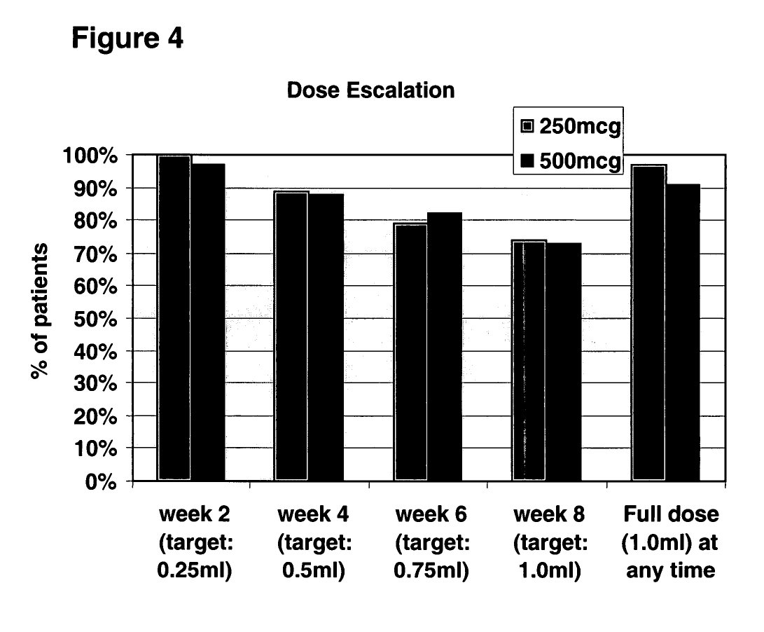

Results: 71 treatment naive RRMS patients were randomized to one of the two treatments (see FIG. 2). Betaseron 500 mcg was well tolerated and safe (see FIG. 3). The dose escalation scheme was as successful in the 500 mcg group as the 250 mcg group, with over 90% of patients attaining the full 500 mcg dose during the course of the study (see FIG. 4). Intermittent dose interruptions were similar for the two groups (4 patients in each treatment arm), but dose reductions were more common in the 500 mcg treatment arm.

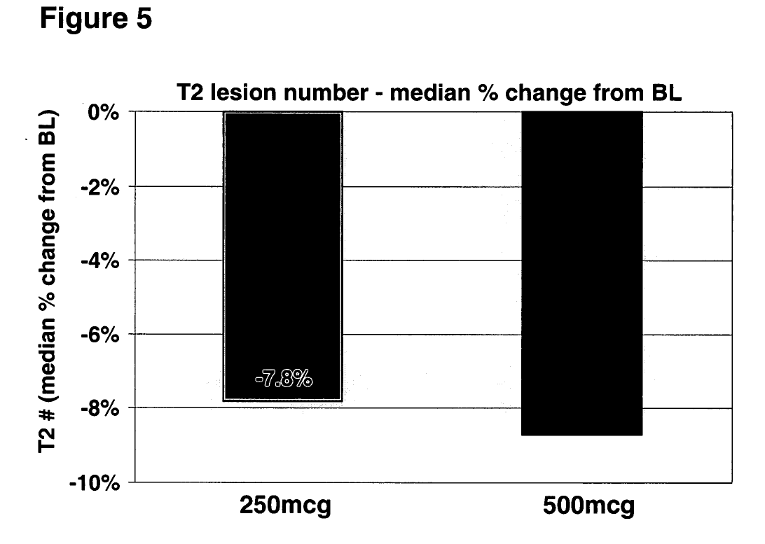

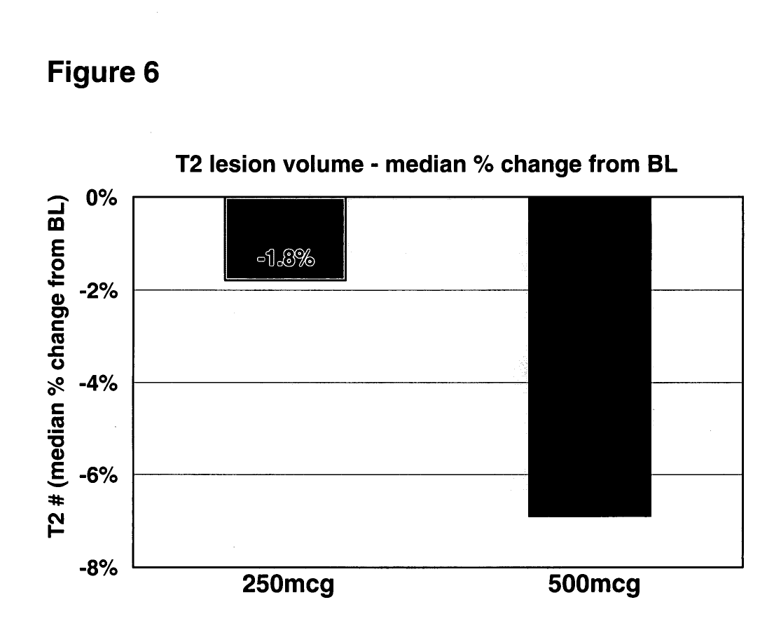

Pre-planned and post-hoc descriptive analyses using MRI measurements showed: median percent change in T2 lesion volume from baseline was −6.9% in the 500 mcg IFN-β 1b group versus −1.8% in the 250 mcg group (T2 lesion number −8.7% versus −7.8%) see FIGS. 5 and 6); median Gd-enhancing lesion volume and number at week 12 was 0 in both groups (baseline volume 0 mm³ versus 13 mm³; baseline number 0 versus 1). Change in mean Gd-enhancing lesion number was −90% in the 500 mcg group versus −70% in the 250 mcg group (volume −96% versus −93%). Median number of newly active lesions at week 12 was 0 in both groups (mean ±SD: 0.8±1.1 versus 1.7±3.8).

Conclusion: The results of this study demonstrate the safe, well-tolerated, and positive trend towards beneficial effects of the administration of a 500 mcg subcutaneous dose IFN-β 1bₛₑᵣ₁₇ eod to patients with RRMS. In particular, using MRI parameters to monitor the effects of higher doses in the treatment of patients with MS, the findings of this study indicate a positive trend towards a beneficial effect of 500 mcg IFN-β 1b as compared to the currently approved 250 mcg dose.

1. A pharmaceutical composition having interferon-beta (IFN-β) activity and comprising a therapeutically effective amount of an isolated IFN-β mutein for treatment of multiple sclerosis (MS),
wherein said therapeutically effective amount is in a range that is greater than 375 mcg to at least about 500 mcg, and
wherein said IFN-β mutein has a cysteine at position 17 deleted or replaced by a neutral amino acid.

2. The pharmaceutical composition according to claim 1, wherein said therapeutically effective amount is at least about 500 mcg to at least about 625 mcg.

3. The pharmaceutical composition according to claim 1, wherein said therapeutically effective amount is at least about 450 mcg to at least about 550 mcg.

4. The pharmaceutical composition according to claim 1, wherein said therapeutically effective amount is at least about 475 mcg to at least about 525 mcg.

5. The pharmaceutical composition according to claim 1, wherein said therapeutically effective amount is about 500 mcg.

6. The pharmaceutical composition according to claim 1, wherein said neutral amino acid is selected from a group consisting of serine, threonine, glycine, alanine, valine, leucine, isoleucine, histidine, tyrosine, phenylalanine, tryptophan, and methionine.

7. The pharmaceutical composition according to claim 1, wherein said neutral amino acid is serine.

8. The pharmaceutical composition according to claim 1, wherein said therapeutically effective amount is about 500 mcg and said neutral amino acid is serine.

9. The pharmaceutical composition according to claim 1, wherein said IFN-β mutein lacks an N-terminal methionine.

10. The pharmaceutical composition according to claim 1, wherein said IFN-β mutein is Betaseron®.

11. The pharmaceutical composition according to claim 1, wherein said pharmaceutical composition is a stabilized, human serum albumin-free (HSA-free) pharmaceutical composition.

12. The pharmaceutical composition according to claim 9, wherein said IFN-β mutein is substantially monomeric and solubilized in a low-ionic-strength formulation.

13. The pharmaceutical composition according to claim 10, wherein said low-ionic-strength formulation is a solution having a pH from about 2 to about 5, and an ionic strength from about 1 to about 100 mM.

14. The pharmaceutical composition according to any one of claims 1-11, wherein said IFN-β mutein is a human IFN-β mutein.

15. A method of treating a patient for multiple sclerosis comprising administering to said patient the pharmaceutical composition according to any one of claims 1-11.

16. The method according to claim 13, wherein said IFN-β mutein is a human IFN-β mutein.
